(19) United States
(12) Patent Application Publication
(10) Pub. No.: US 2017/0119430 A1
(43) Pub. Date: May 4, 2017

(54) PERITONIAL DIALYSIS PORT APPARATUS

(71) Applicants: Amir Keshvari, TEHRAN (IR); Mohammad Reza Nassajian, TEHRAN (IR)

(72) Inventors: Amir Keshvari, TEHRAN (IR); Mohammad Reza Nassajian, TEHRAN (IR)

(21) Appl. No.: 15/407,211

(22) Filed: Jan. 16, 2017

(51) Int. Cl. A61B 17/34; A61M 39/24; A61M 1/28

(52) U.S. Cl. CPC A61B 17/3415; A61M 1/28; A61B 17/3421; A61M 39/24; A61B 17/3494

(57) ABSTRACT

The embodiments herein provide a port apparatus for rectus sheath tunneling. The apparatus comprises a mandarin for creating tissue tract when advanced through patient's body. Mandarin comprises a sharp shaft and a mandarin cap. The shaft is conically tapered at distal end to avoid damage to peritoneum tissues. The proximal end of mandarin is attached with a mandarin cap. A cannula overlaying mandarin is provided for administering medication, inserting surgical instrument or drawing fluids when placed in patient's body. Cannula comprises hollow tube for reaching peritoneal cavity. Cannula comprises head assembly detachably attached to proximal end of hollow tube for providing grasping surface to surgeon. The head assembly comprises cannula cap and cup for holding unidirectional valve for preventing backflow of air. Mandarin extends beyond the length of cannula at distal end for guiding cannula through tissue tract. Mandarin cap prevents complete passage of mandarin through cannula during procedure.

### BACKGROUND

Technical field

The embodiments herein are generally related to the field of medical procedures. The embodiments herein are particularly related to apparatus and method for surgical placement of a catheter into the body of a patient. The embodiments herein are more particularly related to a peritoneal dialysis port apparatus and associated methods of using the port apparatus for surgical procedures such as insertion of peritoneal dialysis catheters into the peritoneal cavity.

Description of the Related Art

Peritoneal dialysis (PD) is a reliable and accepted treatment modality for patients with end-stage renal disease (ESRD). Functional long-term access to the peritoneal cavity is the key to a successful dialysis. A peritoneal dialysis catheter is mandatory for such procedures. The access of peritoneal cavity is established by creating a controlled cutaneo-peritoneal pathway and passing a catheter through the pathway and the abdominal wall. Such procedures usually lead to issues of infectious complications and mechanical problems resulting from blockage of the catheter tip by omentum, displacement of the intraperitoneal section of the catheter to a position of poor function, pericannular leakage of the dialysate fluid from the abdominal wall and hernia resulting from abdominal wall weakening at the catheter insertion site.

Moreover, these mechanical problems further require operative procedures to rescue the old catheter or replacement of the catheter with a new one and loss of chance of having the peritoneal dialysis as renal replacement therapy with a permanent transfer to hemodialysis. Thus, oblique cranio-caudal passage of the dialysis catheter through the abdominal wall has been recommended to maintain pelvic orientation of the catheter and to reduce the risk of omental entrapment and catheter tip migration. Similarly, rectus sheath tunneling helps to decrease the rate of catheter flow dysfunction. The tunneling requires implantation of the catheter through a long rectus sheath tunnel to the peritoneal cavity.

Additionally, several techniques have been proposed for insertion of PD catheters. The PD catheters are inserted into the peritoneal cavity using blind percutaneous, open, or laparoscopic techniques. The laparoscopic technique has some advantages over other techniques including direct vision and ensuring a right positioning of the catheter within the pelvic cavity. Compared to the open implantation of the catheter by an open laparotomy incision, the laparoscopic methods lead to a reduced incidence of flow dysfunction and peri-cannular leaks. The laparoscopic methods provide a precise placement of the catheter resulting in a less tissue disruption.

Therefore, the laparoscopic approach is considered to be the best option for making a downward rectus sheath tunneling. In this approach, the catheters are implanted into the peritoneal cavity in a midway between umbilicus and pubis under laparoscopic view. The suitable place considered for positioning a tip of the catheter in the peritoneal cavity is a deep pelvic area. For this purpose, a port apparatus is used to help surgeons for making a long downward tunnel above rectus sheath and peritoneum. If the catheter enters the peritoneal cavity perpendicularly and without the long downward tunnel, it can be rotated to reach every side of the abdomen. While the catheter is inserted into the peritoneal cavity with downward tunnel and the catheter movement is restricted, the catheter is not rotated to reach the upper parts or lateral sides of the abdomen easily. Also, the catheter is still prone for rotation in any direction within the abdominal cavity due to unavailability of inadequate fixation of intramural segment. Further, these catheters are at a risk of peri-catheter leakages and hernias.

Although there are general improvements associated with lateral placement, abdominal wall tunneling and laparoscopically directed percutaneous insertion of the catheters using the laparoscopic view, the outcomes are inconsistent as far as a success rate is concerned. These wide ranges of output resulting from a lack of uniformity and availability of dedicated implantation tools/port apparatus and methodology, forces the doctors to use the existing tools. Thus, the surgeons usually use oversized laparoscopic port cannulas with trocar blades, which are dangerous for the abdominal tissues and require considerable modifications, to perform a laparoscopic percutaneous insertion of the catheter and muscular tunneling. A large number of port apparatuses are available for use in a wide variety of applications. This reduces reproducibility and makes the establishment of a standard methodology for peritoneal dialysis catheter implantation difficult. Abdominal wall tunneling using open dissection disrupts the tissues required for creation a sufficient length. An excessive tissue dissection during open dissection increases the risk of pericannular leaks and hernias. This also limits the length of an abdominal wall tunnel. The risk of hemorrhage, leaks and hernias also increases with oversized or bladed laparoscopic ports. The length of rectus sheet tunneling is also limited with an overlying plastic sleeve for the radial expansion. The sleeve gets kinked within the tissue tract, when the guide needle is removed, thereby resulting in a prevention of a safe insertion of the port cannula.

Hence, there is a need for a dedicated port apparatus specifically designed to provide an improved and safe access to the peritoneal cavity for implantation of peritoneal dialysis catheters. Yet there is a need for a specialized PD port apparatus for creating a downward long oblique rectus sheet tunnel through the abdominal wall without hemorrhage and significant tissue damage. Further, there is a need for a PD port apparatus that preserves the pneumoperitoneum after gas insufflations. Still further, there is a need for a simple, accurate and safe PD port apparatus having minimal dimensions for passing the catheter.

The above-mentioned shortcomings, disadvantages and problems are addressed herein and which will be understood by reading and studying the following specification.

### OBJECTS OF THE EMBODIMENTS

The primary object of the embodiments herein is to provide a dedicated port apparatus specifically designed to provide an improved and safe access to the peritoneal cavity for implanting the peritoneal dialysis catheters.

Another object of the embodiments herein is to provide a specialized PD port apparatus for creating a downward long oblique rectus sheet tunnel through the abdominal wall without hemorrhage and significant tissue damage.

Yet another object of the embodiments herein is to provide a port apparatus that is easy to handle for the surgeons and helps to make an appropriate rectus sheath tunneling with minimal complications.

Yet another object of the embodiments herein is to provide a simple and accurate PD port apparatus having minimal dimensions to pass the catheter.

Yet another object of the embodiments herein is to provide a port apparatus that preserves the pneumoperitoneum after gas insufflations.

Yet another object of the embodiments herein is to provide a port apparatus that prevents a backflow of air during the rectus sheath tunneling procedure by using a unidirectional pneumatically competent plastic valve.

Yet another object of the embodiment herein is to create a tissue tract by preventing a severe damage and rupture of peritoneum tissues by using a tapered tip mandarin.

Yet another object of the embodiment herein is to provide a port apparatus and a method for surgical implantation of peritoneal dialysis catheter with proper rectus sheath tunneling under the vision of laparoscopic camera into the peritoneal cavity.

Yet another object of the embodiment herein is to provide a laparoscopic port apparatus for creating a downward rectus sheath tunneling during the implementation of peritoneal dialysis (PD) catheters that leads to a longer survival of catheter and less catheter dysfunction.

Yet another object of the embodiment herein is to provide a port apparatus that is reusable after washing, cleaning, sterilizing or by changing the unidirectional valve.

Yet another object of the embodiment herein is to provide a port apparatus comprising of a unidirectional valve that resumes its shape after deformation from the passage of the mandarin and dialysis catheter.

Yet another object of the embodiment herein is to provide a port apparatus that is easy to grasp and convenient to hold for surgeons using thumb and fingure during the rectus sheath tunneling procedure.

These and other objects and advantages of the embodiments herein will become readily apparent from the following detailed description taken in conjunction with the accompanying drawings.

### SUMMARY

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The various embodiments herein provide a laparoscopic peritoneal dialysis port apparatus for rectus sheath tunneling. The apparatus comprises a mandarin configured for creating a tissue tract when advanced through a patient's body to perform rectus sheath tunneling. The mandarin comprises a sharp shaft and a mandarin cap. The sharp shaft is conically tapered at a distal end of the mandarin to avoid a severe damage to the peritoneum tissues and a proximal end of the mandarin is attached with a mandarin cap. The port apparatus also comprises a cannula overlaying the mandarin and configured for administering medication, inserting a surgical instrument or drawing off fluids when placed in a patient's body. The cannula comprises a hollow tube configured for reaching the peritoneal cavity during a rectus sheath tunneling through a long oblique tunnel in abdominal wall. The cannula also comprises a head assembly detachably attached to the proximal end of the hollow tube and configured for providing a grasping surface to the surgeon during rectus sheath tunneling. The head assembly comprises a cannula cap and a cup configured for holding a unidirectional valve for preventing backflow of air during the procedure. The mandarin is extended beyond the length of the cannula at the distal end of the mandarin for guiding the cannula through the tissue tract and the mandarin cap prevents a complete passage of mandarin through the cannula during the procedure.

According to an embodiment herein, the cannula cap further comprises a furrow at an inner rim of a distal part and a central hollow at a proximal par. The diameter of the central hollow is sufficient enough to pass the catheter and associated cuffs.

According to an embodiment herein, the cup further comprises a furrow at an outer rim of a proximal part and at an inner rim of a distal part. The furrow at the proximal part is configured for connecting the cup to the cannula cap by twisting and the furrow at the distal part is configured for connecting the head assembly with the hollow tube.

According to an embodiment herein, the cup further comprises a proximal hollow and a distal hollow. The diameter/size/dimension of the proximal hollow is larger than that of the distal hollow The proximal hollow is configured for holding the unidirectional valve and the distal hollow is configured for providing a passage for a catheter and associated cuffs.

According to an embodiment herein, the distal hollow of the cup comprises an oblique facet at an external rim. The oblique facet is configured for providing a place for surgeon's thumb and fingers while grasping the port apparatus in hand and also while advancing the port apparatus in a proper direction during rectus sheath tunneling.

According to an embodiment herein, an outer diameter of the hollow tube at a distal part is smaller than at the proximal part that results into a toothed surface on the upper part of the hollow tube.

According to an embodiment herein, the unidirectional valve comprises a hole configured for permitting passage of desired items such as mandarin, dialysis catheter and associated components such as cuffs.

According to an embodiment herein, the unidirectional valve is further configured for providing an airtight seal, when the pressure distal to the unidirectional valve exceeds that to the proximal.

According to an embodiment herein, the cannula cap and the cup are detached from the head assembly by twisting and are reusable for the next rectus sheath tunneling procedure by washing, cleaning, sterilizing or by changing the unidirectional valve.

According to an embodiment herein, the mandarin is about 12 cm to 20 cm long and has a diameter of about 6 mm.

According to an embodiment herein, the hollow tube is about 9 cm to 17 cm long and has a diameter of about 7 mm.

According to an embodiment herein, the connections of the head assembly to the hollow tube and that of the cannula cap to the cup are designed using mechanisms that include, but are not limited to, furrow and twisting, slot and hinge configurations, snaps, magnets, magnetic buttons, fasteners, buckles, side-release buckles and the like.

According to an embodiment herein, the port apparatus is manufactured using materials such as plastics, polymers, alloys, resins, metals and the like.

According to an embodiment herein, the unidirectional valve possesses elastic properties configured for allowing the unidirectional valve to resume its shape after deformation from the passage of the mandarin and dialysis catheter.

According to an embodiment herein, a method of using a port apparatus for surgical procedures such as insertion of peritoneal dialysis catheters into the peritoneal cavity is provided. The method comprises the steps of identifying a catheter insertion site and performing skin incision and subcutaneous tissue dissection resulting in the fascia of the rectus abdominal muscle being exposed. A port apparatus is inserted in perpendicular fashion in the abdominal wall through the anterior layer of the muscle fascia and the port apparatus is advanced through the rectus muscle. The tip of the mandarin is monitored using a laparoscope as the mandarin tents down the posterior fascia of the rectus muscle. The port apparatus is advanced with the tip of the mandarin by making downward angle as the port apparatus is slid down the posterior fascia of the rectus muscle. The port apparatus is pushed through the peritoneum into the abdominal cavity when the desired tunnel length within the rectus fascial sheath is achieved. The distal part of hollow tube of the cannula is advanced into the peritoneal space for creating a long oblique tunnel in a craniocaudal direction through the muscular abdominal wall and for promoting pelvic orientation of the catheter. The mandarin is extracted from the cannula while the distal hollow tube remains in the peritoneal space. A peritoneal dialysis catheter having cuffs is advanced using a stylet through the cannula's hollow tube. The stylet is withdrawn temporarily while the peritoneal dialysis catheter is in peritoneal space and disconnects the head assembly of the cannula from the hollow tube. The hollow tube is withdrawn from the abdominal wall while the peritoneal dialysis catheter remains in peritoneal space. The stylet is loaded again into the peritoneal dialysis catheter for proper placement of the tip of the catheter in deep pelvis and of proximal cuff over the peritoneum. The stylet and laparoscopic port apparatus are removed after satisfactory placement of the peritoneal dialysis catheter.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### BRIEF DESCRIPTION OF THE DRAWINGS

The other objects, features and advantages will occur to those skilled in the art from the following description of the preferred embodiment and the accompanying drawings in which:

FIGS. 1A, 1B, and 1C illustrate a perspective view of a port apparatus held by a surgeon during rectus sheath tunneling, according to one embodiment herein.

FIG. 2 illustrates a side sectional view of the port apparatus, according to one embodiment herein.

FIG. 3A illustrates a side sectional view of a head assembly in the port apparatus, according to one embodiment herein.

FIG. 3B illustrates a side sectional view of the hallow tube, according to one embodiment herein.

FIG. 3C illustrates a side sectional view of the cannula, according to one embodiment herein.

FIG. 3D illustrates a side sectional view of the mandarin, according to one embodiment herein.

FIG. 4 illustrates an exploded side sectional side view of the head assembly indicating a cannula cap, unidirectional valve and cup in detached condition, according to one embodiment herein.

FIGS. 5A, 5B, 5C, 5D, 5E, 5F, and 5J illustrate the stepwise procedure of laparoscopic rectus sheath tunneling method performed with the port apparatus, according to one embodiment herein.

FIG. 6 illustrates a flow chart explaining a method of operating a port apparatus for surgical procedures such as insertion of peritoneal dialysis catheters into the peritoneal cavity, according to one embodiment herein.

Although the specific features of the present invention are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a laparoscopic peritoneal dialysis port apparatus for rectus sheath tunneling. The apparatus comprises a mandarin configured for creating a tissue tract when advanced through a patient's body to perform rectus sheath tunneling. The mandarin comprises a sharp shaft and a mandarin cap. The sharp shaft is conically tapered at a distal end of the mandarin to avoid a severe damage to the peritoneum tissues and a proximal end of the mandarin is attached with a mandarin cap. The port apparatus also comprises a cannula overlaying the mandarin and configured for administering medication, inserting a surgical instrument or drawing off fluids when placed in a patient's body. The cannula comprises a hollow tube configured for reaching the peritoneal cavity during a rectus sheath tunneling through a long oblique tunnel in abdominal wall. The cannula also comprises a head assembly detachably attached to the proximal end of the hollow tube and configured for providing a grasping surface to the surgeon during rectus sheath tunneling. The head assembly comprises a cannula cap and a cup configured for holding a unidirectional valve for preventing backflow of air during the procedure. The mandarin is extended beyond the length of the cannula at the distal end of the mandarin for guiding the cannula through the tissue tract and the mandarin cap prevents a complete passage of mandarin through the cannula during the procedure.

According to an embodiment herein, the cannula cap further comprises a furrow at an inner rim of a distal part and a central hollow at a proximal par. The diameter of the central hollow is sufficient enough to pass the catheter and associated cuffs.

According to an embodiment herein, the cup further comprises a furrow at an outer rim of a proximal part and at an inner rim of a distal part. The furrow at the proximal part is configured for connecting the cup to the cannula cap by twisting and the furrow at the distal part is configured for connecting the head assembly with the hollow tube.

According to an embodiment herein, the cup further comprises a proximal hollow and a distal hollow. The diameter/size/dimension of the proximal hollow is larger than that of the distal hollow The proximal hollow is configured for holding the unidirectional valve and the distal hollow is configured for providing a passage for a catheter and associated cuffs.

According to an embodiment herein, the distal hollow of the cup comprises an oblique facet at an external rim. The oblique facet is configured for providing a place for surgeon's thumb and fingers while grasping the port apparatus in hand and also while advancing the port apparatus in a proper direction during rectus sheath tunneling.

According to an embodiment herein, an outer diameter of the hollow tube at a distal part is smaller than at the proximal part that results into a toothed surface on the upper part of the hollow tube.

According to an embodiment herein, the unidirectional valve comprises a hole configured for permitting passage of desired items such as mandarin, dialysis catheter and associated components such as cuffs.

According to an embodiment herein, the unidirectional valve is further configured for providing an airtight seal, when the pressure distal to the unidirectional valve exceeds that to the proximal.

According to an embodiment herein, the cannula cap and the cup are detached from the head assembly by twisting and are reusable for the next rectus sheath tunneling procedure by washing, cleaning, sterilizing or by changing the unidirectional valve.

According to an embodiment herein, the mandarin is about 12 cm to 20 cm long and has a diameter of about 6 mm.

According to an embodiment herein, the hollow tube is about 9 cm to 17 cm long and has a diameter of about 7 mm.

According to an embodiment herein, the connections of the head assembly to the hollow tube and that of the cannula cap to the cup are designed using mechanisms that include, but are not limited to, furrow and twisting, slot and hinge configurations, snaps, magnets, magnetic buttons, fasteners, buckles, side-release buckles and the like.

According to an embodiment herein, the port apparatus is manufactured using materials such as plastics, polymers, alloys, resins, metals and the like.

According to an embodiment herein, the unidirectional valve possesses elastic properties configured for allowing the unidirectional valve to resume its shape after deformation from the passage of the mandarin and dialysis catheter.

According to an embodiment herein, a method of using a port apparatus for surgical procedures such as insertion of peritoneal dialysis catheters into the peritoneal cavity is provided. The method comprises the steps of identifying a catheter insertion site and performing skin incision and subcutaneous tissue dissection resulting in the fascia of the rectus abdominal muscle being exposed. A port apparatus is inserted in perpendicular fashion in the abdominal wall through the anterior layer of the muscle fascia and the port apparatus is advanced through the rectus muscle. The tip of the mandarin is monitored using a laparoscope as the mandarin tents down the posterior fascia of the rectus muscle. The port apparatus is advanced with the tip of the mandarin by making downward angle as the port apparatus is slid down the posterior fascia of the rectus muscle. The port apparatus is pushed through the peritoneum into the abdominal cavity when the desired tunnel length within the rectus fascial sheath is achieved. The distal part of hollow tube of the cannula is advanced into the peritoneal space for creating a long oblique tunnel in a craniocaudal direction through the muscular abdominal wall and for promoting pelvic orientation of the catheter. The mandarin is extracted from the cannula while the distal hollow tube remains in the peritoneal space. A peritoneal dialysis catheter having cuffs is advanced using a stylet through the cannula's hollow tube. The stylet is withdrawn temporarily while the peritoneal dialysis catheter is in peritoneal space and disconnects the head assembly of the cannula from the hollow tube. The hollow tube is withdrawn from the abdominal wall while the peritoneal dialysis catheter remains in peritoneal space. The stylet is loaded again into the peritoneal dialysis catheter for proper placement of the tip of the catheter in deep pelvis and of proximal cuff over the peritoneum. The stylet and laparoscopic port apparatus are removed after satisfactory placement of the peritoneal dialysis catheter.

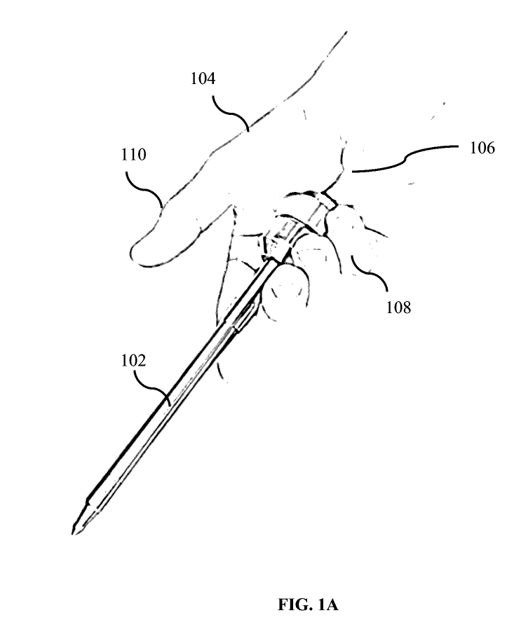

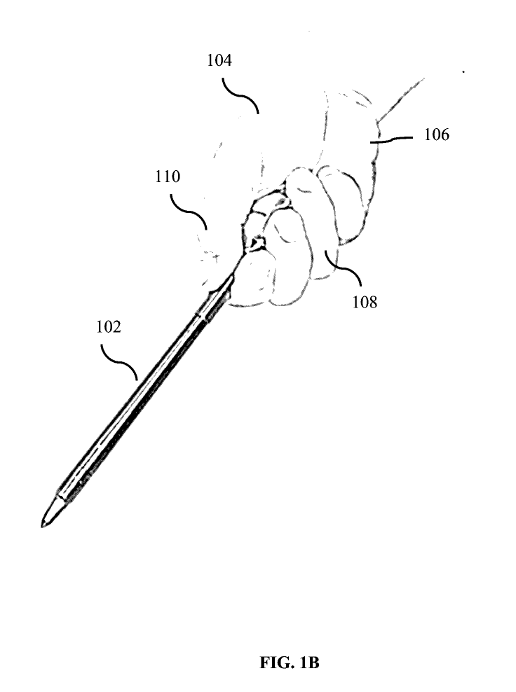

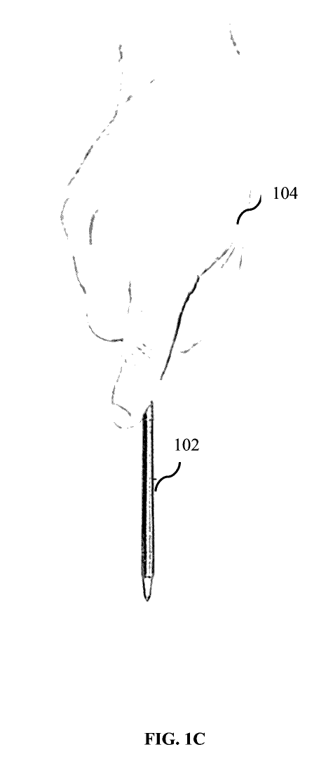

FIGS. 1A-1C illustrate various techniques of holding the port apparatus by a surgeon during rectus sheath tunneling, according to one embodiment herein. With respect to FIG. 1A-C, the laparoscopic PD port apparatus 102 is held by the hand 104 of a surgeon. FIG. 1A shows position of the port apparatus between the palm 106 and fingers 108 of surgeon. FIG. 1B shows position of the thumb 110 and fingers 108 of surgeon when grasping the port apparatus. FIG. 1C shows the procedure-ready state of the port apparatus in the hand of the surgeon.

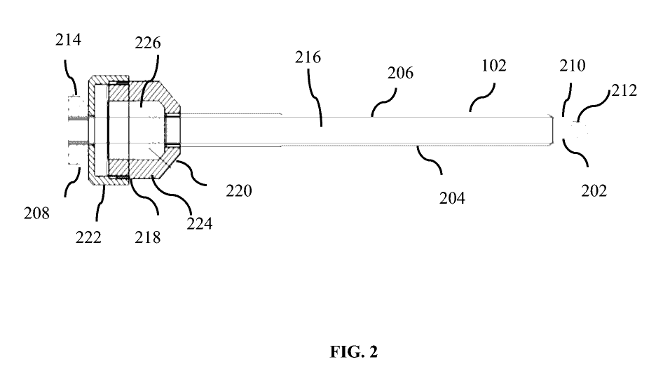

FIG. 2 illustrates a cross-sectional side view of the port apparatus, according to one embodiment herein. With respect to FIG. 2, the port apparatus 102 comprises a mandarin 202, and a cannula 204. The mandarin 202 comprises a sharp shaft 206 that passes through the hollow long axis of the cannula 204. The mandarin comprises a proximal end 208 and a distal end 210. The mandarin 202 is configured for extending beyond the length of the cannula 204 at its distal end 210. The sharp shaft 206 is conically tapered 212 at the distal end 210 of the mandarin 202 to make a safe rectus sheath tunneling. This gradually tapered mandarin tip 212 when advanced through tissue creates a tissue tract and prevents severe damage and rupture of surrounding tissues, such as peritoneum. Compared to standard trocar blades, the defects caused by a tapered tip mandarin 212 are smaller and the risk of hemorrhage is tolerable.

According to an embodiment herein, the mandarin 202 comprises a mandarin cap 214 over the proximal end 208. The mandarin cap 214 is configured for preventing complete passage of the mandarin 202 through the cannula 204.

According to an embodiment herein, the cannula 204 comprises a hollow tube 216 configured for reaching the peritoneal cavity during rectus sheath tunneling through a long oblique tunnel in abdominal wall. The cannula 204 also comprises a head assembly 218 detachably attached to the proximal end 220 of the hollow tube 216. The head assembly 218 is configured for providing a grasping surface to the surgeon during rectus sheath tunneling. The head assembly 218 further comprises a cannula cap 222 and a cup 224 configured for holding a unidirectional valve 226 for preventing backflow of air during the procedure. In one example embodiment, the unidirectional valve 226 is a pneumatically competent plastic valve.

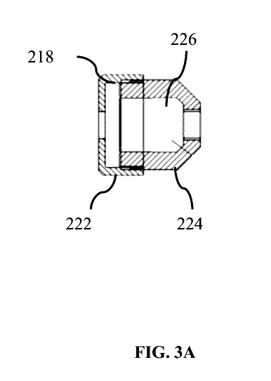

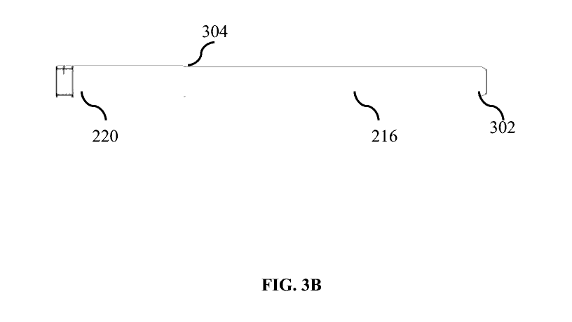

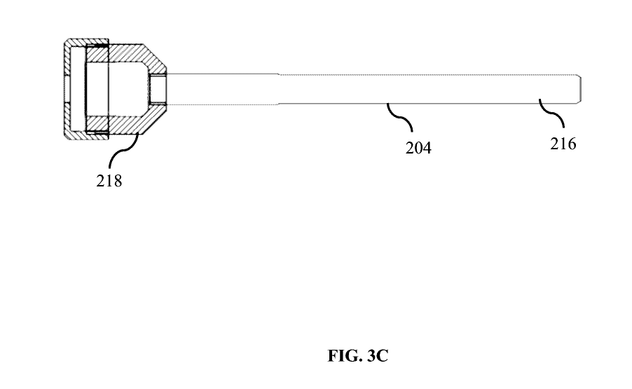

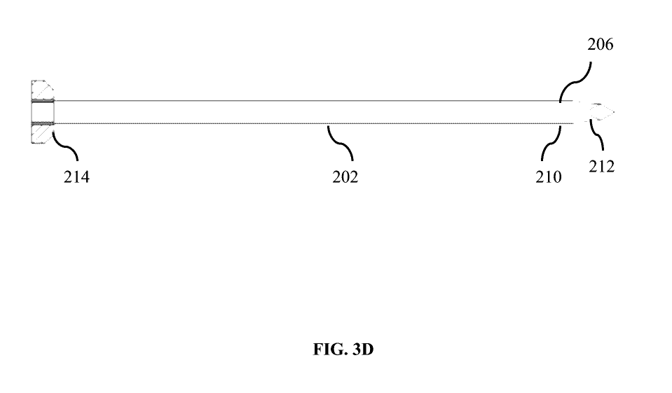

FIG. 3A illustrates a cross sectional side view of the head assembly, according to one embodiment herein. With respect to FIG. 3A, the arrangement of cannula cap 222 and a cup 224 holding a unidirectional valve 226 within the head assembly 218 is shown. In one example embodiment, the head assembly 218 is 3 cm long.

FIG. 3B illustrates a cross sectional side view of the hallow tube, according to one embodiment herein. With respect to FIG. 3B, an outer diameter of the hollow tube 216 at a distal end 302 is smaller than at the proximal end 220 that results into a toothed surface 304 on the upper part of the hollow tube 216. The hollow tube is about 9 cm to 17 cm long and has a diameter of about 7 mm. In one example embodiment, the hollow tube is 13 cm long with an inner diameter of 3 mm and an outer diameter of 7 mm.

According to an embodiment herein, the hollow tube 216 comprises a straight cylindrical pipe and is provided with sufficient length to easily reach the peritoneal cavity through a long oblique tunnel in abdominal wall and for making a proper rectus sheet tunneling. The inner diameter of the hollow tube 216 is fitted for easy passage of the PD catheter with associated cuffs. Here, lesser diameter of hollow tube 216 in distal part produces smaller aperture and lesser trauma to the peritoneum.

FIG. 3C illustrates a cross sectional side view of the cannula, according to one embodiment herein. With respect to FIG. 3C, the cannula 204 comprising of the hollow tube 216 and head assembly 218 attached together is shown. In one example embodiment, the head assembly 218 is detachably attached to the hollow tube 216 during the procedure.

FIG. 3D illustrates a cross sectional side view of the mandarin, according to one embodiment herein. With respect to FIG. 3D, the mandarin 202 comprising of sharp shaft 206 conically tapered 212 at the distal end 210 and the mandarin cap 214 is shown. The mandarin 202 is about 12 cm to 20 cm long and has a diameter of about 6 mm. In one example embodiment, the mandarin is about 16 cm long and about 6 mm in diameter.

According to an embodiment herein, the connections of the head assembly 218 to the hollow tube 216 and that of the cannula cap 222 to the cup 224 are designed using mechanisms that include, but are not limited to, furrow and twisting, slot and hinge configurations, snaps, magnets, magnetic buttons, fasteners, buckles, side-release buckles and the like. The port apparatus 102 is manufactured using materials such as plastics, polymers, alloys, resins, metals and the like.

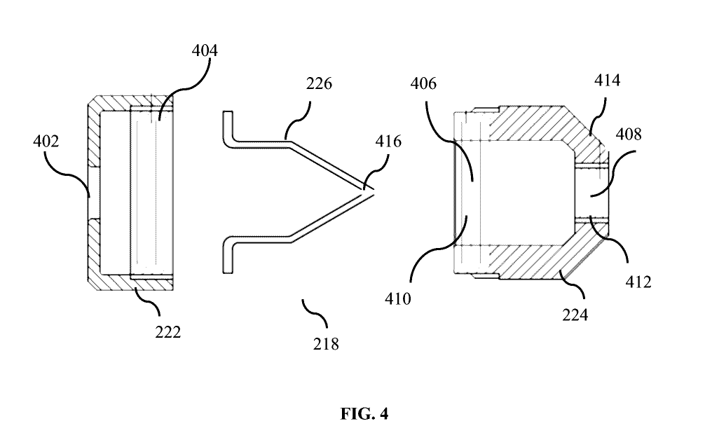

FIG. 4 illustrates an expanded cross-sectional side view of the head assembly depicting cannula cap, unidirectional valve and cup in detached condition, according to one embodiment herein. With respect to FIG. 4, the head assembly 218 comprising of cannula cap 222 and cup 224 holding a unidirectional valve 226 in between for preventing backflow of the air during the procedure is shown. The cannula cap 222 comprises a central hollow 402 in proximal part. The diameter of the central hollow 402 is sufficient for passing the catheter and associated cuffs. A furrow 404 is provided at an inner rim of the distal part of the cannula cap 222 for connecting it to the cup 224 by twisting.

According to an embodiment herein, the cup 224 comprises a proximal hollow 406 and a distal hollow 408. The proximal hollow 406 is larger as compared to the distal hollow 408 and has a diameter suitable for holding the unidirectional valve 226. The distal hollow 408 has a diameter suitable for passage of catheter and associated cuffs. The cup 224 comprises a furrow 410 and 412 at an outer rim of a proximal part and at an inner rim of a distal part respectively. The furrow 410 at the proximal part is configured for providing connection of the cup 224 to the cannula cap 222 by twisting and the furrow 412 at the distal part is configured for providing connection of the head assembly 218 with the hollow tube 216. In one example embodiment, the cannula cap 222 and cup 224 are not disassembled during the procedure. The cannula cap 222 and cup 224 of the head assembly 218 are disassembled easily by twisting for washing, cleaning or for changing the unidirectional valve 226.

According to an embodiment herein, the head assembly 218 is designed so as to be easily grasped by the hand of a surgeon during the procedure. The distal hollow 408 of the cup 224 comprises an oblique facet 414 at an external rim. The oblique facet 414 is configured for providing a place for surgeon's thumb and fingers while grasping the port apparatus in hand and also while advancing the port apparatus in a proper direction during rectus sheath tunneling.

According to an embodiment herein, the unidirectional valve 226 comprises a hole 416 configured for permitting passage of desired items such as mandarin, dialysis catheter and associated components such as cuffs. The unidirectional valve 226 is configured for providing an airtight seal when the pressure distal to the unidirectional valve exceeds that to the proximal. The unidirectional valve 226 possesses elastic properties configured for allowing the unidirectional valve 226 to resume its shape after deformation from the passage of the mandarin 202 and dialysis catheter.

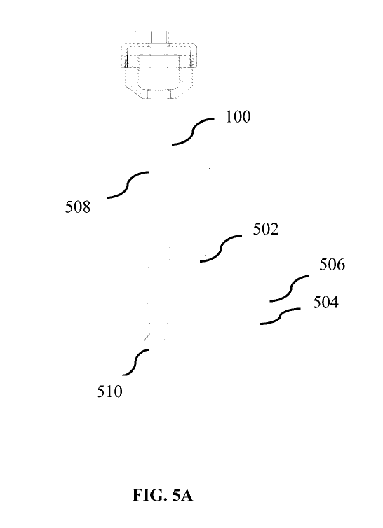

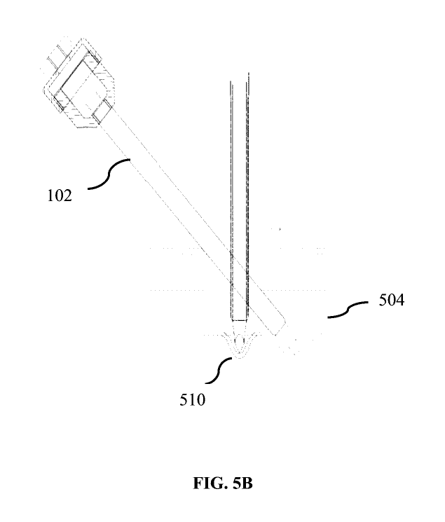

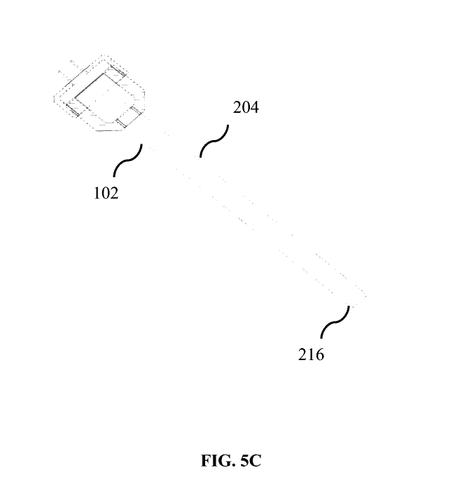

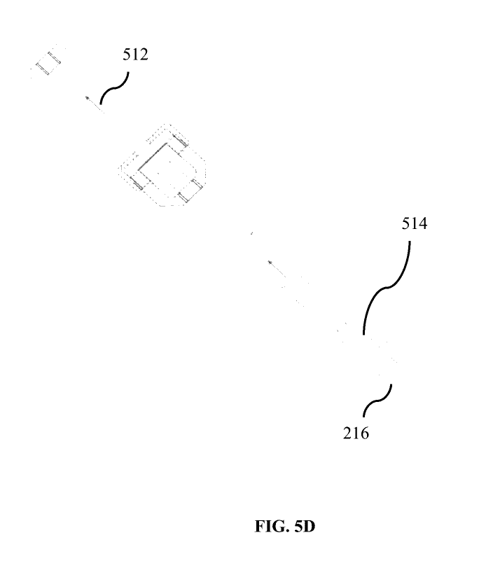

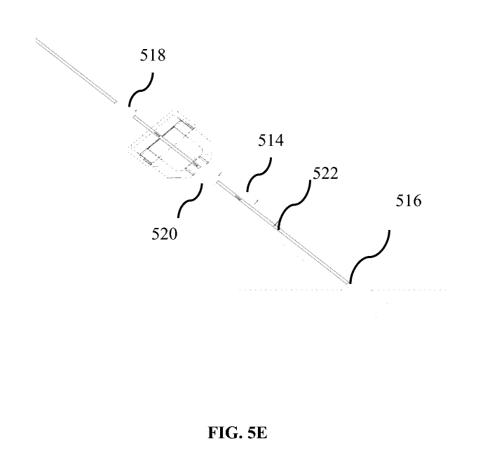

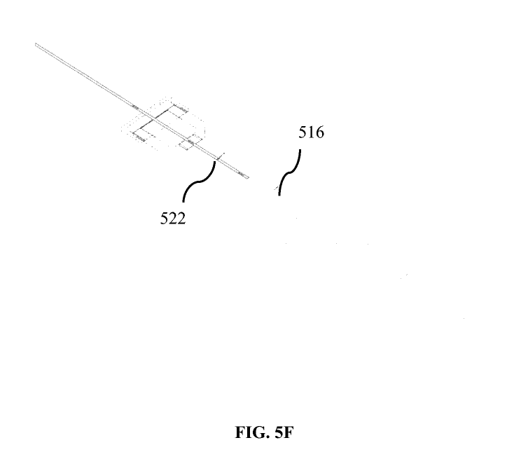

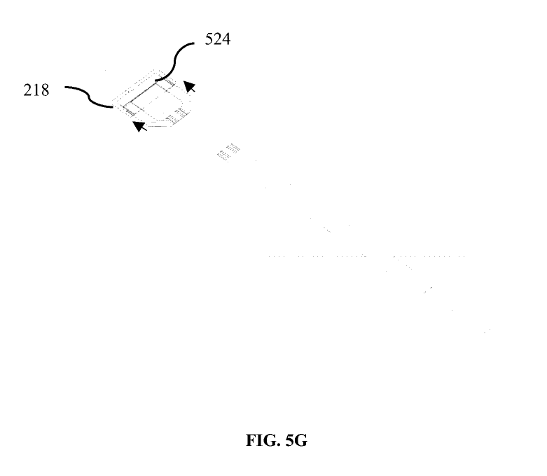

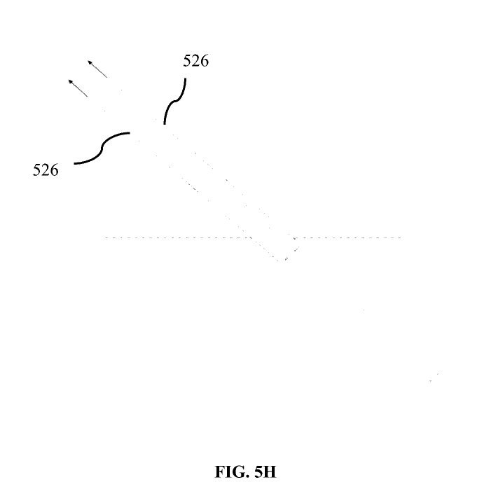

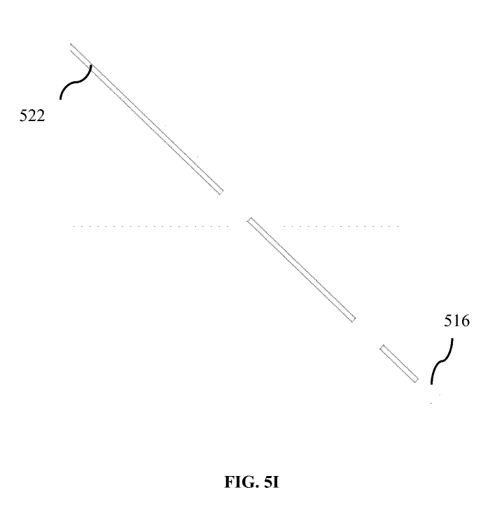

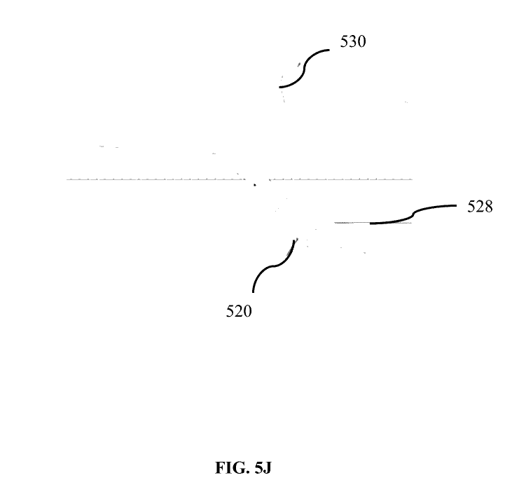

FIGS. 5A-5J illustrate the stepwise procedure of laparoscopic rectus sheath tunneling using the port apparatus, according to one embodiment herein. With respect to FIG. 5A-5J, on completion of gas insufflation a laparoscopic camera (not shown) is inserted at an abdominal wall location remote from the designated peritoneal dialysis catheter insertion site using standard laparoscopic surgical methods. With respect to FIG. 5A, at the designated catheter insertion site after skin incision and subcutaneous tissues dissection 502, the fascia of the rectus abdominis muscle 50 is exposed. The port apparatus 100 is inserted perpendicular to the abdominal wall through the anterior layer of the muscle fascia 506. The port apparatus is advanced through the rectus muscle in a perpendicular fashion 508. The tip of the mandarin 212 is easily visible through the laparoscope (not shown) as the mandarin tents down the posterior fascia 510 of the rectus muscle.

According to an embodiment herein, the port apparatus 102 is then angled downward to the pelvis and advanced with the tip of the mandarin 212 monitored laparoscopically as the mandarin is easily slid down the posterior fascia 510 of the rectus muscle 504 as shown in FIG. 5B. When the tunnel length within the rectus fascial sheath reaches desired range (preferably about 4 cm to 6 cm) then the port apparatus is pushed through the peritoneum into the abdominal cavity. The distal part of hollow tube 216 of the cannula 204 is advanced into the peritoneal space as shown in FIG. 5C. The port apparatus 102 creates a long oblique tunnel in a craniocaudal direction through the muscular abdominal wall and promotes pelvic orientation of the catheter. Here, compared to the catheter that passes perpendicularly through the abdominal wall the potential range of motion of the catheter is reduced.

According to an embodiment herein, the mandarin 202 is then extracted (512) from the cannula 204 while the distal hollow tube 216 remains (514) in the peritoneal space as shown in FIG. 5D. After straightening, the peritoneal dialysis catheter 516 having cuffs 518 and 520 is advanced with a stylet 522 (FIG. 5E) through the cannula's hollow tube 216 (FIG. 5F) under laparoscopic view.

According to an embodiment herein, while the catheter through the port apparatus is in peritoneal space, the stylet 522 is temporarily withdrawn (FIG. 5F) and the head assembly 218 of the cannula 204 is disconnected from hollow tube 216 and is taken out (524) from distal part of the catheter 516 (FIG. 5G). Then, the hollow tube 216 is taken out (526) from the abdominal wall while the catheter remains in peritoneal space as shown in FIG. 5H. The catheter 516 is again loaded by the stylet 522 (FIG. 5I) for proper placement of the tip of the catheter in deep pelvis (not shown) and that of proximal cuff 520 over the peritoneum (528) as shown in FIG. 5J. This maneuver is monitored with the laparoscope to confirm proper position of proximal cuff 520 over peritoneum. After satisfactory placement of the dialysis catheter 516, the stylet 522 is withdrawn. The catheter is exited through a small skin incision, the laparoscopic instruments are removed and the wounds are closed 530 using standard surgical techniques for implanting peritoneal dialysis catheters.

According to an embodiment herein, the laparoscopic PD port apparatus 102 contemplates as a part of a catheter insertion surgical kit that in addition to this port apparatus comprises of appropriate catheter, stylet and other associated surgical components. This kit is provided as a single sterile package or individual components of the kit are packaged and provided individually in sealed sterile packages as part of a larger kit.

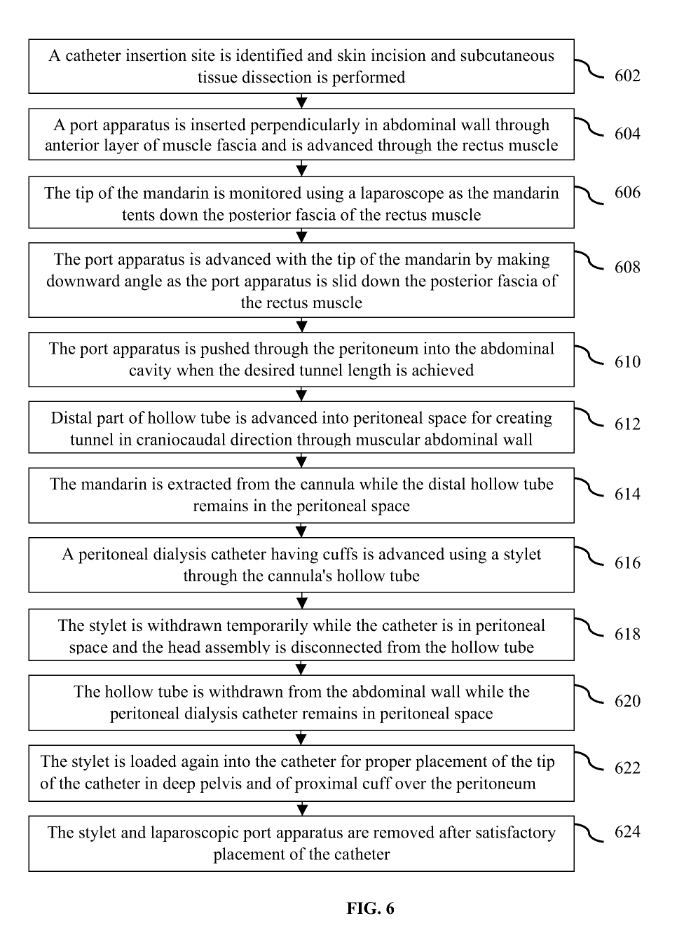

FIG. 6 illustrates a method of using a port apparatus for surgical procedures such as insertion of peritoneal dialysis catheters into the peritoneal cavity, according to one embodiment herein. With respect to FIG. 6, a catheter insertion site is identified and skin incision and subcutaneous tissue dissection is performed resulting in the fascia of the rectus abdominal muscle being exposed (602). A port apparatus is inserted in perpendicular fashion in the abdominal wall through the anterior layer of the muscle fascia and advanced through the rectus muscle (604). The tip of the mandarin is monitored using a laparoscope as the mandarin tents down the posterior fascia of the rectus muscle (606). The port apparatus is advanced with the tip of the mandarin by making downward angle as the port apparatus is slid down the posterior fascia of the rectus muscle (608). The port apparatus is pushed through the peritoneum into the abdominal cavity when the desired tunnel length within the rectus fascial sheath is achieved (610). The distal part of hollow tube of the cannula is advanced into the peritoneal space for creating a long oblique tunnel in a craniocaudal direction through the muscular abdominal wall and for promoting pelvic orientation of the catheter (612). The mandarin is extracted from the cannula while the distal hollow tube remains in the peritoneal space (614). A peritoneal dialysis catheter having cuffs is advanced using a stylet through the cannula's hollow tube (616). The stylet is withdrawn temporarily while the peritoneal dialysis catheter is in peritoneal space and disconnecting the head assembly of the cannula from the hollow tube (618). The hollow tube is withdrawn from the abdominal wall while the peritoneal dialysis catheter remains in peritoneal space (620). The stylet is loaded again into the peritoneal dialysis catheter for proper placement of the tip of the catheter in deep pelvis and of proximal cuff over the peritoneum (622). The stylet and laparoscopic port apparatus are removed after satisfactory placement of the peritoneal dialysis catheter (624).

Therefore, the proposed PD port apparatus provides a proper port for laparoscopic implantation of the peritoneal dialysis catheter. The port apparatus is safe and easy to handle that helps surgeons to make an appropriate rectus sheath tunneling with minimal complications. The port apparatus of the invention is made of stainless steel material to prevent rusting of the particles. It consists of several parts, which are all detachable and can be assembled easily during the procedure. The head assembly of the port apparatus is designed so as to be easily grasped by the hand of the surgeon during the procedure and to make proper RSHT. Insertion of a one-way elastic valve in the proposed port apparatus makes backflow of the insufflated gas impossible from the peritoneal cavity during the passage of the PD catheter through the apparatus and helps in maintaining an adequate pneumoperitoneum.

Moreover, even in obese patients, the long length of the apparatus makes the rectus sheath tunneling with a proper length possible and prevents peri-catheter fluid leakage during the subsequent peritoneal dialysis process. Additionally, using laparoscopic approach, under direct vision, the deep cuffs are positioned within the rectus sheet in a proper location. The material that is used for manufacturing has the advantage of being reusable. Easy handling of the port apparatus during the procedure and during backward removal is another major advantage of the proposed invention.

This invention is utilized for the laparoscopic implantation of PD catheters under local anesthesia using minimal or no sedation. Also, by making use of this port apparatus any intraoperative complications are avoided during the laparoscopic procedure such as hollow viscus perforation, intra-abdominal bleeding, pre-peritoneal hematoma, or uncontrolled perforation of the peritoneum.

The port apparatus with some modification is used for patients with refractory ascites by decompressing the fluid with a tube that shunts the peritoneal fluid to the venous system. The placement of the abdominal tube is facilitated by this port apparatus. Laparoscopic insertion of feeding tube or decompressing tube into the stomach or small intestine is also facilitated with this port apparatus. The port apparatus is also used to laparoscopically assist in placing a drain into a hollow viscus, e.g., gallbladder or bladder, or tube decompression of a fluid collection such as an abscess or lymphocele.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims. It is also to be understood that the following claims are intended to cover all of the generic and specific features of the embodiments described herein and all the statements of the scope of the embodiments which as a matter of language might be said to fall there between.

What is claimed is:

1. A laparoscopic peritoneal dialysis port apparatus for rectus sheath tunneling, the apparatus comprising:
a mandarin configured for creating a tissue tract when advanced through a patient's body to perform a rectus sheath tunneling, wherein the mandarin comprises a sharp shaft and a mandarin cap, and wherein the sharp shaft is conically tapered at a distal end of the mandarin to avoid damage to peritoneum tissues, and wherein a proximal end of the mandarin is attached with a mandarin cap;
a cannula overlaying the mandarin and configured for administering medication, inserting a surgical instrument or drawing off fluids when placed in a patient's body, and wherein the cannula comprises a hollow tube configured for reaching a peritoneal cavity during rectus sheath tunneling through a long oblique tunnel in an abdominal wall;
a head assembly detachably attached to the proximal end of the hollow tube and configured for providing a grasping surface to a surgeon during the rectus sheath tunneling, and wherein the head assembly comprises a cannula cap and a cup configured for holding a unidirectional valve for preventing backflow of air during a surgical procedure; and
wherein the mandarin is extended beyond a length of the cannula at the distal end of the mandarin for guiding the cannula through a tissue tract and wherein the mandarin cap is configured to prevent a complete passage of mandarin through the cannula during the surgical procedure.

2. The port apparatus according to claim 1, wherein the cannula cap further comprises a furrow at an inner rim of a distal part and a central hollow at a proximal part, wherein the diameter of the central hollow is sized enough to pass the catheter and associated cuffs.

3. The port apparatus according to claim 1, wherein the cup further comprises a furrow at an outer rim of a proximal part and at an inner rim of a distal part, wherein the furrow at the proximal part is configured for connecting the cup to the cannula cap by a twisting operation and wherein the furrow at the distal part is configured for connecting the head assembly with the hollow tube.

4. The port apparatus according to claim 1, wherein the cup further comprises a proximal hollow and a distal hollow, and wherein the proximal hollow is larger in dimension than the distal hollow, and wherein the proximal hollow is configured for holding the unidirectional valve, and wherein the distal hollow is configured for providing a passage for a catheter and associated cuffs.

5. The port apparatus according to claim 4, wherein the distal hollow of the cup comprises an oblique facet at an external rim, and wherein the oblique facet is configured for providing a place for surgeon's thumb and fingers while grasping the port apparatus in hand and also while advancing the port apparatus in a proper direction during a rectus sheath tunneling.

6. The port apparatus according to claim 1, wherein an outer diameter of the hollow tube at a distal part is smaller than that at the proximal part, and wherein the proximal part has a toothed surface on an upper part of the hollow tube.

7. The port apparatus according to claim 1, wherein the unidirectional valve comprises a hole configured for permitting a passage of desired items, and wherein the desired items includes mandarin, dialysis catheter and associated components, and wherein the associated components include a cuffs.

8. The port apparatus according to claim 1, wherein the unidirectional valve is further configured for providing an airtight seal, when a pressure at a distal end of the unidirectional valve exceeds than that of the proximal end.

9. The port apparatus according to claim 1, wherein the cannula cap and the cup are detached from the head assembly by a twisting operation, and wherein the cannula cap and the cup are reused for a next rectus sheath tunneling procedure by washing, cleaning, sterilizing or by changing the unidirectional valve.

10. The port apparatus according to claim 1, wherein the mandarin has a length of 12 cm to 20 cm and a diameter of 6 mm.

11. The port apparatus according to claim 1, wherein the hollow tube has a length of 9 cm to 17 cm and has a diameter of about 7 mm.

12. The port apparatus according to claim 1, wherein the head assembly is connected to the hollow tube and the cannula cap is connected to the cup through a connecting interface or connector mechanism, and wherein the connecting interface or connector mechanism, is selected from a group consisting of a furrow, slot and hinge configurations, snaps, magnets, magnetic buttons, fasteners, buckles, side-release buckles.

13. The port apparatus according to claim 1, wherein the port apparatus is made of materials selected from a group consisting of plastics, polymers, alloys, resins, and metals.

14. The port apparatus according to claim 1, wherein the unidirectional valve has elastic properties configured for allowing the unidirectional valve to resume original shape after deformation from a passage of the mandarin and dialysis catheter.
